(19) United States
(12) Patent Application Publication
(10) Pub. No.: US 2019/0269489 A1
(43) Pub. Date: Sep. 5, 2019

(54) Male Incontinence Clamp

(71) Applicant: Rainer Kuenzel, Hunt, TX (US)

(72) Inventor: Rainer Kuenzel, Hunt, TX (US)

(21) Appl. No.: 16/106,692

(22) Filed: Aug. 21, 2018

Related U.S. Application Data
(60) Provisional application No. 62/606,338, filed on Sep. 19, 2017.

(51) Int. Cl. A61F 2/00

(52) U.S. Cl. CPC A61F 2/0054; A61F 2230/0013; A61F 2220/0083; A61F 2220/0091

(57) ABSTRACT

A clamping device that protects male patients, who suffer from incontinence, from urinal leakage, which is designed to make wearing it more comfortable, makes attaching it to the patients penis simplified, and has foam rubber cushioning devices attached by “VELCRO”, to make them exchangeable and therefore easily replaceable by new ones to avoid replacing the entire device, when such cushioning parts wear out or become too unsightly after long-time usage, and can no longer be kept clean by washing them with soap and water.

This Patent application is based on the Provisional Application U.S. 62/606,338, filed Sep. 19, 2017.

### DESCRIPTION OF THE DRAWING FIGURES

FIG. 1 is a cross section of the invention as indicated in FIG. 2;

FIG. 2 is a bottom view of FIG. 1;

FIG. 3 is a left side view of FIG. 1;

FIG. 4 is a right side view of FIG. 1;

FIG. 5 is a cross section, like FIG. 1, in an open position;

FIG. 6 is a right side view of FIG. 5;

FIG. 7 is a side elevation view of the device in closed position;

FIG. 8 is a cross section as indicated in FIG. 7;

FIG. 9 is a side elevation view of the device in partially open position;

FIG. 10 is an alternative design detail in cross section as indicated in FIG. 9;

FIG. 11 is a side elevation view with an alternative detail;

FIG. 12 is a cross section view of an alternative design as indicated in FIG. 13;

FIG. 13 is a top view of FIG. 12;

FIG. 14 is a left side view of FIG. 12;

FIG. 15 is a right side view of FIG. 12;

FIG. 16 is a right side view of the device in an open position;

FIG. 17 is a side elevation view of the clamp in an installed position.

### SUMMARY OF THE INVENTION

The problem most older men have, and that for various medical reasons, is the inability to control the release of bodily fluids, mainly urine, which in most cases cannot be treated and must be endured by the suffering individual for the rest of his life.

It is therefore important to make the management of such a condition as comfortable as possible.

One method often used is the installation of a permanent catheter into the urethra and let the urine flow through it continually into a plastic bag, which must be worn strapped to the upper thigh of the person, and must be emptied out periodically.

Another method is the permanent catheter which has a valve at its outer end, which must be opened every 2 or so hours to empty out the bladder.

The least embarrassing method is the use of a clamp, which simply is placed over the end of the penis; pressure is applied to squeeze the urethra shut to a point where no urine can escape. Such clamps must be removed to urinate, which is next to impossible to do in a standing position by a urinal and requires a sit-down procedure.

In addition to that, these clamps must be cleaned frequently with soap and water. If they are equipped with cushioning foam rubber, cleaning that material is difficult because it tends to soak up every fluid it comes into contact with; it also tends to deform when used over a longer time, which makes frequent replacement of the entire clamp necessary.

It is also important that a clamp like this has no protruding corners, which tend to painfully poke into a users upper thigh or other body parts in its close vicinity.

It is therefore important to provide a clamp which eliminates all the above mentioned shortcomings, and makes this embarrassing but necessary business as comfortable as possible.

The present invention aims to accomplish that.

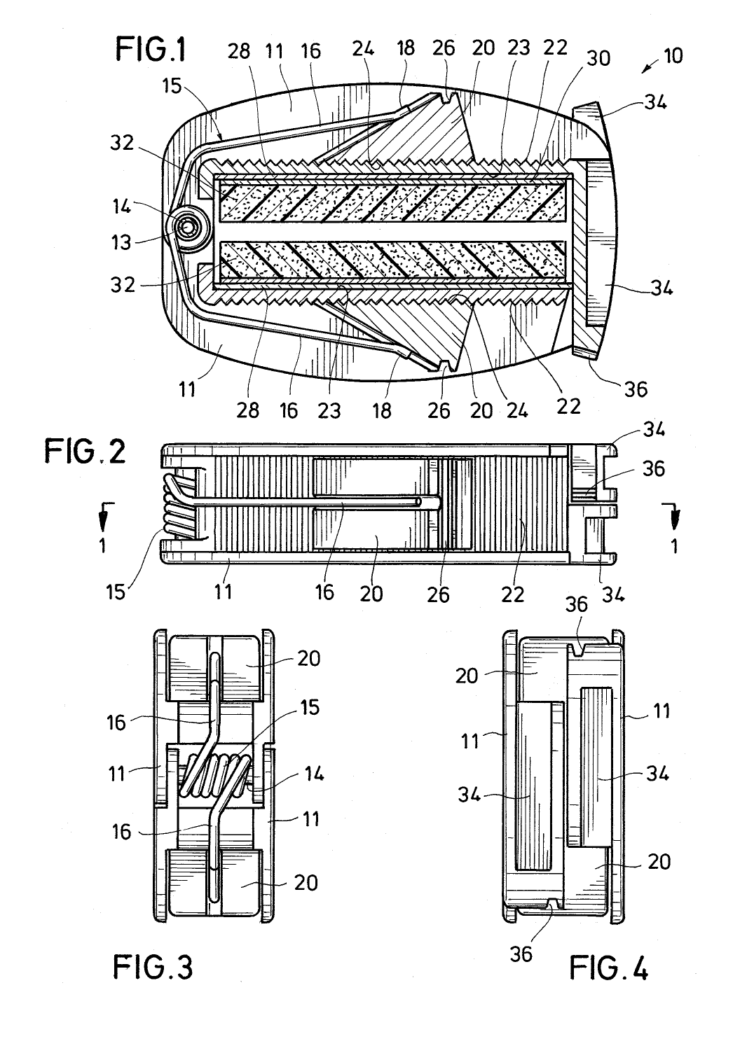

Referring to FIGS. 1 to 4, Clamp (10) consists of two identical U-shaped halves (11), which are hinged together at hinge point (13) by pin (14). Surrounding hinge pin (14) is a torque spring (15), with extending ends (16) applying pressure to urge clamp halves (11) toward each other. Wedges (20) can be placed between spring ends (18) and the outer surfaces (22) of the clamp halves. Wedges (20) can be moved away or toward the spring to spread the spring ends more or less apart to increase or decrease the pressure to close the clamp. Surfaces (22) of the clamp halves are provided with serrations, so are the contacting surfaces (24) of the wedges to avoid unintended movement of the wedges.

Notches (26) in the outermost surface of the wedges serve to move the wedges by using a thumb nail or any flat object.

The inner surfaces (23) of the clamp halves are equipped with hook-type “VELCRO” strips (28), which serve to engage loop-type “VELCRO” strips (30), which are part of cushioning bars (32). These bars can be made of foam rubber or a soft silicone material, which would not soak up fluids. These cushioning bars can easily be removed and re-installed after cleaning.

Eventually though, the cushioning bars—no matter what material they are made of—will be deteriorated to the point where they need to be replaced. This too is facilitated by the “VELCRO” attachment, and the entire device will not have to be discarded.

The ends of the clamp halves opposite to the hinge side are each other overlapping extensions (34) at a 90 degree angle relative to the main body of the clamp halves and are half the width of the clamp halves.

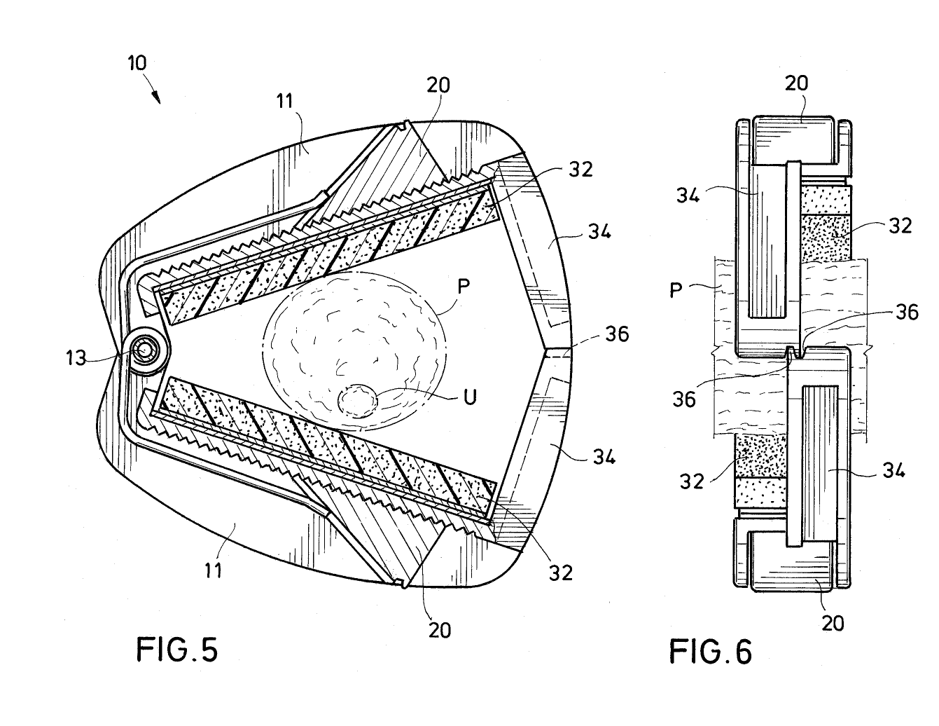

Referring now to FIGS. 5 and 6, the clamp halves are shown spread apart against the spring pressure. The extensions (34) are deflected somewhat toward each other and are resting against each other by engaging notches (36). In this position enough distance between the clamp halves is provided to slip the device over the end of the man's penis (P).

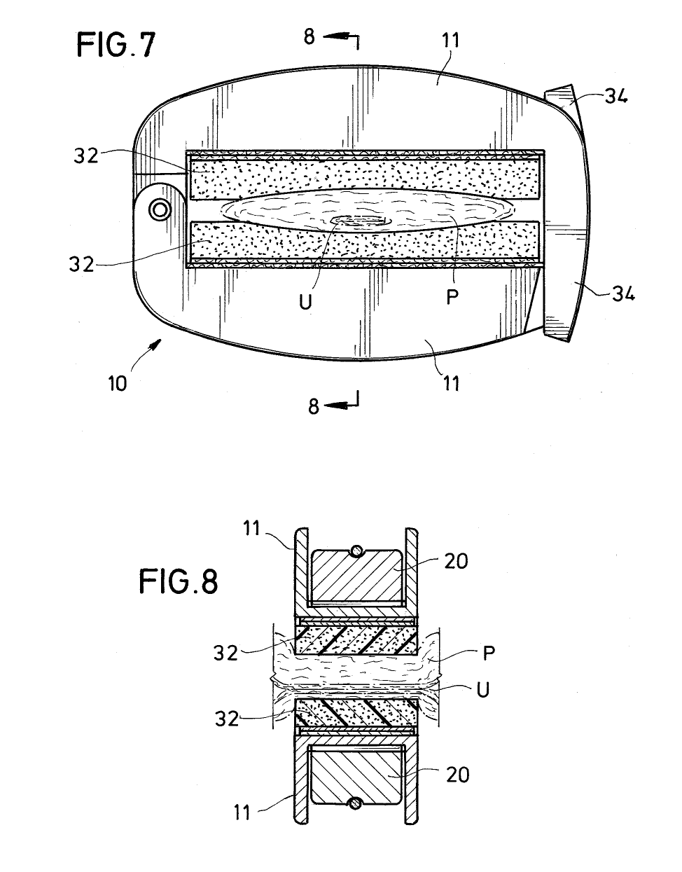

By disengaging notches (36), the clamp halves are allowed to be pushed against each other by spring (15), thus squeezing penis (P) to the point of closing down the urethra (U) and preventing urinal leakage, as illustrated in FIGS. 7 and 8. In case some leakage still occurs, pressure of the torque spring can be increased by moving one or both wedges (20) as described above.

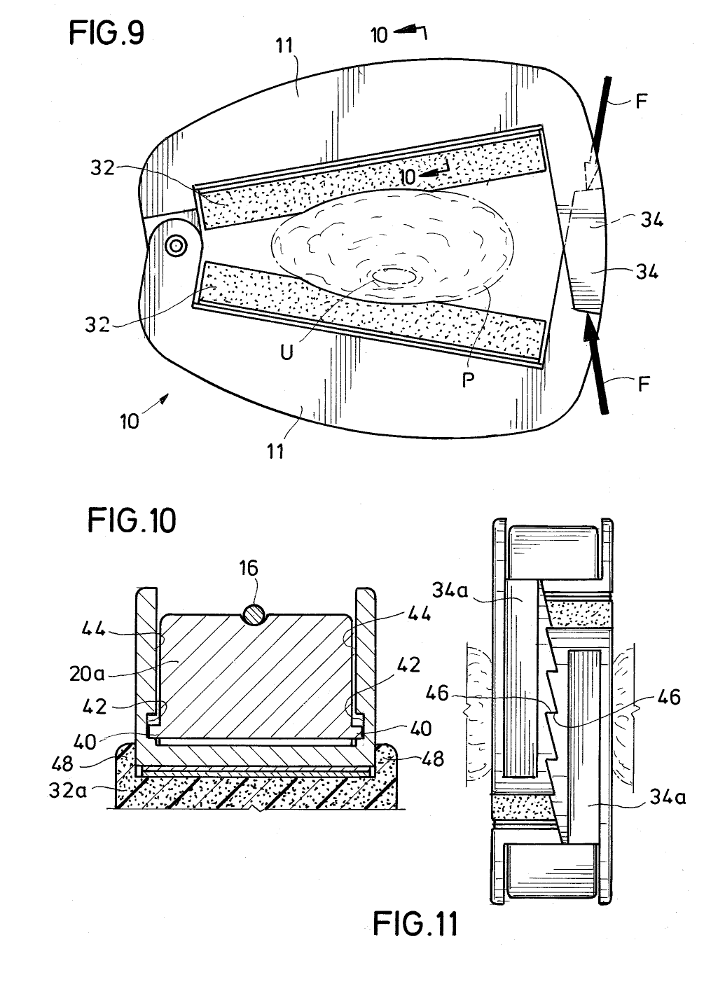

Referring to FIG. 9, the operation of urinating is illustrated. Without having to remove the clamp from the penis (P), the user applies a slight pressure (F) to the clamp extensions (34), thus opening the space between the clamp haves enough to relax the compression of the penis to the point of opening the urethra sufficiently to allow urine to flow.

Alternative design details are illustrated in FIGS. 10 and 11. FIG. 10 shows a wedge (20a) with protrusions (40) at the bottom edges, which engage slots (42) along the sidewalls (44) of the clamp halves.

Furthermore an alternative form of the cushioning bar is illustrated, where the sides (48) of the cushion bar (32a) extend outward to cover the edges of the “VELCRO” strips.

In FIG. 11 clamp extensions (34a) are equipped with steps (46) and (47), allowing the user to open the clamp to a sufficient space between halves for urination, and then arrest the position of the halves by flexing the extensions against each other to engage the steps.

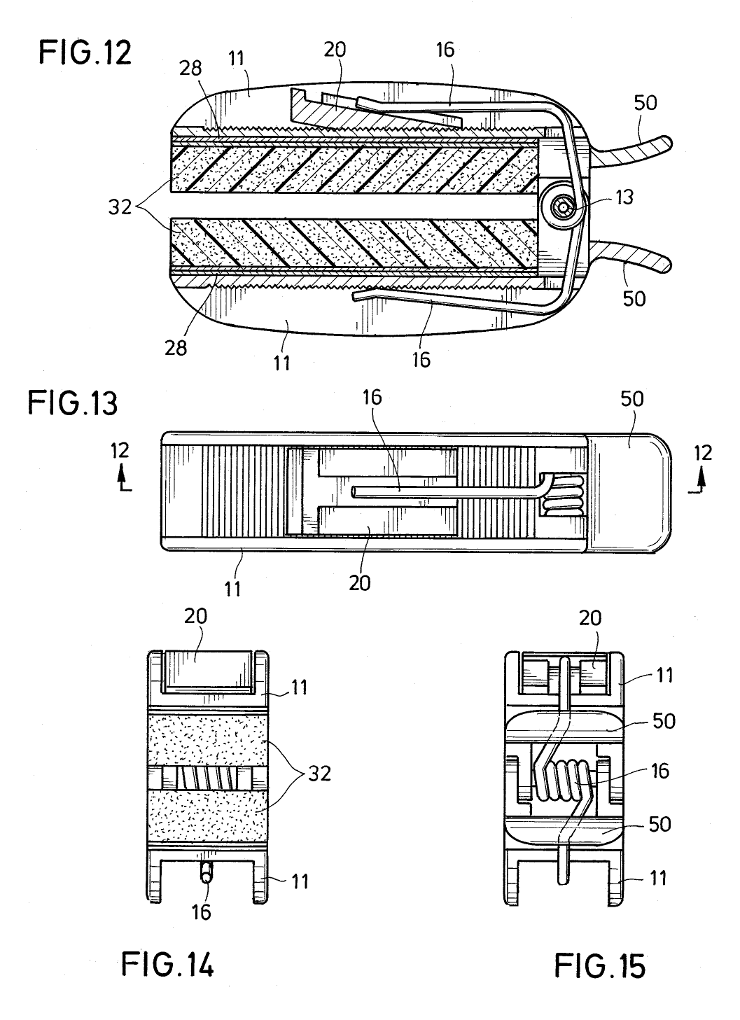

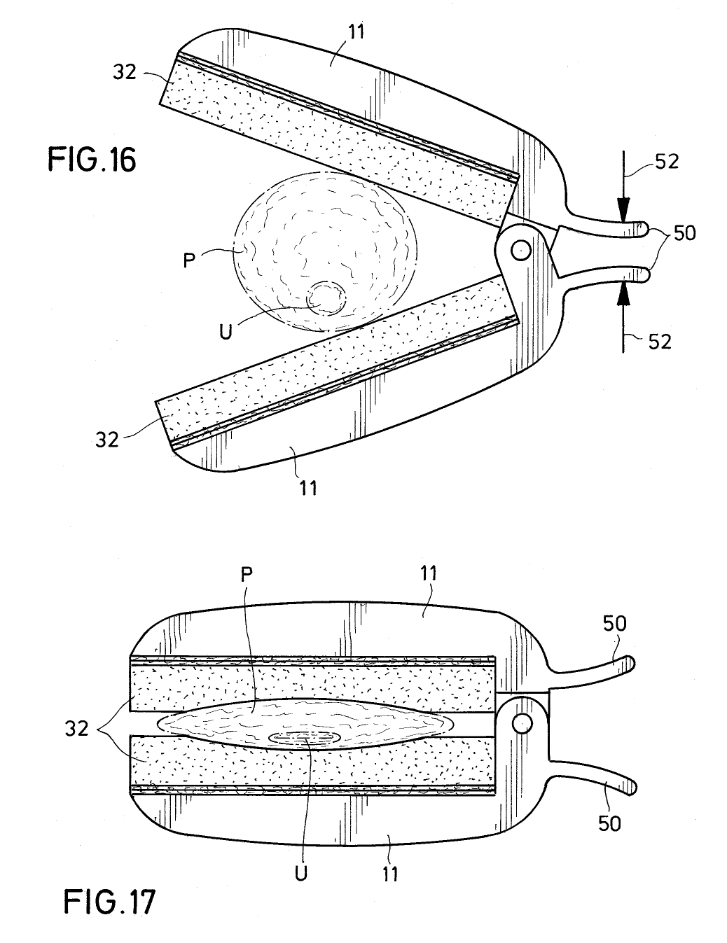

FIG. 12 shows a variation of the clamp, which consists of the same parts as described before, except here the overlapping extensions (34), as shown in FIG. 1, are eliminated. Instead, extensions (50) outside the hinge side are added to open the clamp by applying pressure (52) to the extensions (50).

I claim:

1. An incontinence clamp for male patients, consisting of two U-shaped clamp arms, which are at one end connected by a hinge, connected by a hinge pin, further consisting of a torque spring, which surrounds and is held in place by said hinge pin, the extended ends of said spring being bent to press down on the said U-shaped clamp arms.

2. A clamp of claim 1, further consisting of wedges being held between the bottom of said U-shaped arms and the ends of said spring ends, such that the clamp closing torque of said spring is increased.

3. The contact surfaces between said wedges and the bottom of said clamp arms, being serrated to prevent said wedges from slipping out of a selected position,

4. Said clamp further consisting of foam rubber or similar soft material cushion devices being attached to the opposing surfaces of the U-shaped arms by “VELCRO” strips to make them removable,

5. The clamp arms being equipped at their ends opposite the hinge side by levers at a 90 degree angle relative to the clamp arms and overlapping each other, each lever long enough to protrude far enough beyond each other so pressure can be applied to their ends,

6. A clamp as in claims 1, 2, 3 and 4, with levers extending outward beyond the hinge to facilitate opening the clamp by applying pressure to them.
